(19) United States
(12) Patent Application Publication
(10) Pub. No.: US 2023/0242953 A1
(43) Pub. Date: Aug. 3, 2023

(54) Engineered Bacterial Strain and Method of Use for One-Pot Vitamin C Synthesis

(71) Applicant: WiSys Technology Foundation, Inc., Madison, WI (US)

(72) Inventor: Paul Bruce Schweiger, Onalaska, WI (US)

(21) Appl. No.: 18/008,820

(22) PCT Filed: Jun. 7, 2021
(86) PCT No.: PCT/US2021/036127

§ 371 Date: Dec. 7, 2022

Related U.S. Application Data
(60) Provisional application No. 63/036,052, filed on Jun. 8, 2020.

(51) Int. Cl. C12P 17/04; C12N 15/52; C12N 15/74; C12N 1/20; C12N 9/04

(52) U.S. Cl. CPC C12P 17/04; C12N 1/20; C12N 9/0006; C12N 15/52; C12N 15/74; C12Y 101/01006; C12Y 101/99012; C12Y 101/99021

(57) ABSTRACT

An engineered bacterial strain expressing dehydrogenases capable of oxidizing D- sorbitol to 2-keto-gulonic acid is provided by the present invention. Methods of using same in a one-pot synthesis of L-ascorbic acid (vitamin C) are also described.

### CROSS-REFERENCE TO RELATED APPLICATIONS

This application claims the benefit of U.S. Provisional Application No. 63/036,052, filed Jun. 8, 2020, which is incorporated herein by reference in its entirety.

### STATEMENT REGARDING FEDERALLY SPONSORED RESEARCH OR DEVELOPMENT

Not Applicable.

### FIELD OF THE INVENTION

This invention relates generally to the synthesis of L-ascorbic acid (vitamin C). In particular, this invention is directed to a one-pot synthesis of L-ascorbic acid (vitamin C) facilitated by a single, engineered bacterial strain capable of oxidizing D-sorbitol to 2-keto-gulonic acid.

### BACKGROUND OF THE INVENTION

More than 200,000 tons of vitamin C are produced annually, which contribute to a vitamin supplement industry that tops $30 billion annually. Vitamin C is presently the number three vitamin supplement within this industry. Aside from the supplement market, vitamin C finds use in the pharmaceutical, nutritional/nutraceutical, and foodstuff industries, substantially adding to its overall market value.

Presently, vitamin C is produced in the commercial setting using a two-step fermentation process. The first step in the commercial process is the conversion of D-sorbitol to L-sorbose and is catalyzed by the polyol dehydrogenase (SldBA) of Gluconobacter oxydans. L-Sorbose is then transferred to a second fermenter where it is converted to 2-keto-gulonic acid by Ketogulonicigenium vulgare by a sorbose/sorbosone dehydrogenase (KVU _2142) and a sorbosone dehydrogenase (KVU _0095). The resulting 2-keto-gulonic acid is then chemically converted to vitamin C. In terms of enzymatic activity, critical protein complexes required for the conversion of D-sorbitol to 2-keto-gulonic acid are absent from both G. oxydans and K. vulgare. Use of the two different microbes allows the conversion of substrates to proceed as the two microbes’ expression patterns, taken together, provide the necessary enzymatic activities, but those activities take place in two separate spaces at distinct times.

While the two-step process is effective, a one-step process would be much more efficient in the industrial setting. Production in a single fermenter is predicted to increase the production yield by allowing the two existing fermenters to be run simultaneously for the full biological production scheme, limiting the space required and increasing fermenter turnover time (i.e. improving the space-time yield). Accordingly, there exists a long felt need in vitamin C production for innovative solutions facilitating efficient single fermenter processes (a.k.a. “one-pot synthesis”).

### SUMMARY OF THE INVENTION

The present invention is directed to a one-pot synthesis of vitamin C providing various advantages over prior methods and is expected to save both production time and expense. Accordingly, the invention provides in a first aspect a one-pot method for synthesizing L-ascorbic acid (vitamin C). Such a method includes steps of: (a) oxidation of D-sorbitol to L-sorbose by a polyol dehydrogenase; (b) oxidation of the L-sorbose by a sorbose dehydrogenase and sorbosone dehydrogenase to 2-keto-gulonic acid; (c) conversion of the 2-keto-gulonic acid to L-ascorbic acid (vitamin C); and (d) isolation of said L-ascorbic acid (vitamin C) synthesized in the method; wherein: the polyol dehydrogenase, sorbose dehydrogenase, and sorbosone dehydrogenase are expressed by a single, engineered bacterial strain, the bacterial strain solely-capable of the steps (a)-(b) whereby D-sorbitol is oxidized to 2-keto-gulonic acid; and the steps (a)-(b) are carried out in a single fermentation vessel.

In preferred embodiments, the polyol dehydrogenase is polyol dehydrogenase (SldBA) of Gluconobacter oxydans, and the preferred sorbose dehydrogenase and sorbosone dehydrogenase are sorbose dehydrogenase (KVU _2142) and sorbosone dehydrogenase (KVU_0095) of Ketogulonicigenium vulgare.

Engineered bacterial strains according to the invention are preferably an engineered Ketogulonicigenium sp. strain, more preferably a Ketogulonicigenium vulgare strain.

In certain embodiments, the engineered bacterial strain is a Ketogulonicigenium vulgare strain naturally expressing the sorbose dehydrogenase and sorbosone dehydrogenase, preferably sorbose dehydrogenase (KVU_2142) and sorbosone dehydrogenase (KVU_0095), and engineered to express the polyol dehydrogenase, a heterologous polyol dehydrogenase, preferably a polyol dehydrogenase (SldBA) of Gluconobacter oxydans.

The conversion step (c) is preferably carried out in the same fermentation vessel as steps (a)-(b).

In another aspect, the invention encompasses an engineered Ketogulonicigenium sp., preferably a Ketogulonicigenium vulgare strain, comprising a heterologous polyol dehydrogenase capable of oxidizing D-sorbitol to L-sorbose.

The engineered Ketogulonicigenium vulgare strain preferably includes the heterologous polyol dehydrogenase (SldBA) of Gluconobacter oxydans, and, more preferably, further expresses a native sorbose dehydrogenase (KVU_2142) and native sorbosone dehydrogenase (KVU _0095), said native dehydrogenases collectively capable of converting L-sorbose to 2-keto-gulonic acid.

In yet another aspect, the invention provides a heterologous expression system for oxidation of D-sorbitol to 2-keto-gulonic acid in a one-pot synthesis. Such a system includes a bacterial strain engineered to express a polyol dehydrogenase, a sorbose dehydrogenase, and a sorbosone dehydrogenase, wherein the bacterial strain is solely-capable of oxidizing D-sorbitol to 2-keto-gulonic acid; and a fermentation vessel containing the engineered bacterial strain and operating under fermentation conditions facilitating oxidation of D-sorbitol to 2-keto-gulonic acid by the bacterial strain.

The bacterial strain is preferably an engineered Ketogulonicigenium sp., preferably a Ketogulonicigenium vulgare strain, wherein, in certain embodiments, the engineered Ketogulonicigenium vulgare strain naturally expresses the sorbose dehydrogenase and sorbosone dehydrogenase and is engineered to express the polyol dehydrogenase, said polyol dehydrogenase being a heterologous polyol dehydrogenase. The heterologous polyol dehydrogenase is preferably from Gluconobacter oxydans.

As can be appreciated, the present invention is directed to an engineered bacterial strain and method of using the same in a one-pot synthesis of L-ascorbic acid (vitamin C). The present invention utilizes a one-pot approach to increase production turnover and limit the production steps to a single space. Accordingly, the present invention provides substantial reductions in the total cost of vitamin C production as compared to previous methods, including one-pot syntheses utilizing two or more bacterial strains. Additional objects, features and advantages of the present invention will become apparent after review of the specification and claims.

### BRIEF DESCRIPTION OF THE DRAWINGS

FIG. 1(A) illustrates current vitamin C production scheme requiring two fermentation steps.

FIG. 1(B) depicts a one-pot vitamin C synthesis according to the invention. The polyol dehydrogenase is derived from G. oxydans. Incorporation into K. vulgare allows the production process to be completed in one step.

FIG. 2 illustrates a cloning scheme to engineer a G. oxydans polyol dehydrogenase (SldBA) heterologous expression system according to the invention.

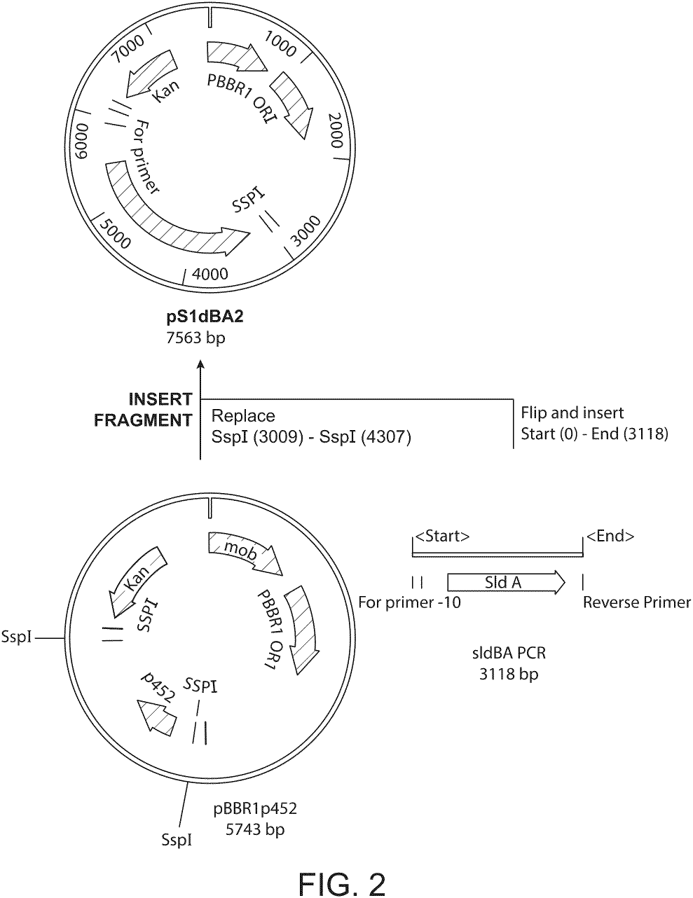

FIG. 3 shows validation data confirming the successful selection of engineered K. vulgare after conjugation with E. coli.

FIG. 4 illustrates a pSldBA expression vector containing a native Gluconobacter sp. sldBA gene, including the 5′-UTR elements required for expression.

### DETAILED DESCRIPTION OF THE INVENTION

### I. In General

Before the present materials and methods are described, it is understood that this invention is not limited to the particular methodology, and materials described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. The Invention

Acetic acid bacteria are well known for their ability to incompletely oxidize their carbon sources under normal growth conditions. The products of these oxidations are released directly into the medium. This characteristic is exploited industrially to produce many important industrial and consumer products, including vinegar, antidiabetic drugs (e.g., miglitol), and vitamin C.

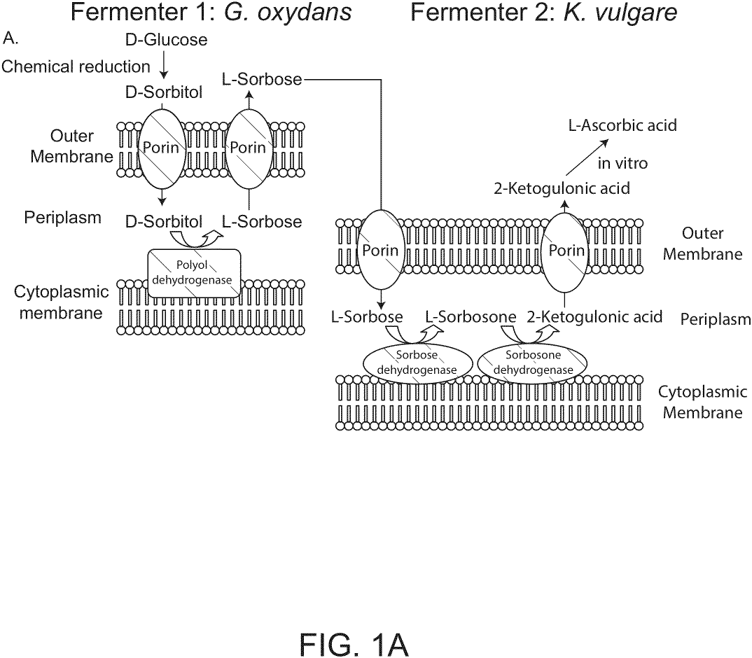

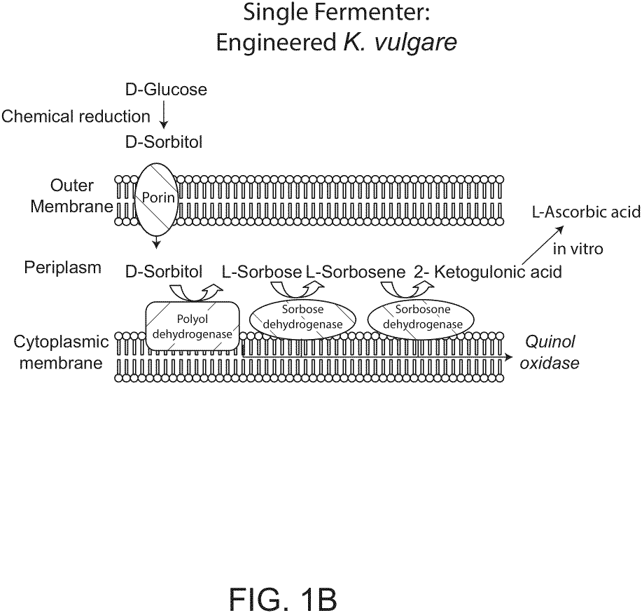

Vitamin C is produced in large volumes industrially (over 200,000 tons annually) in a two-step fermentation process that involves using two separate fermentation vessels (FIG. 1(A)). The first fermentation step oxidizes D-sorbitol to L-sorbose and is catalyzed by the polyol dehydrogenase (SldBA) of the acetic acid bacterium Gluconobacter oxydans. L-Sorbose is further oxidized in a separate fermenter by the sorbose dehydrogenase and sorbosone dehydrogenase of Ketogulonicigenium vulgare into 2-keto-gulonic acid, which is then chemically converted to vitamin C.

While the above two-step process accomplishes the goal of vitamin C production, a one-step process would be much more efficient industrially. Production in a single fermenter is predicted to increase the production yield by allowing the two existing fermenters to be run simultaneously for the full biological production scheme, limiting the space required and increasing fermenter turnover time (i.e. improving the space-time yield).

The construction of a one-pot vitamin C production system has great potential to reduce production cost by reducing the space-time yield. Specifically, by reducing the number of fermentation steps for production, both fermenters in the current system can be used simultaneously with no or little cost in redesigning the production facility. This is important since the purchasing and redesign of industrial fermentation systems is a major cost consideration when adopting new technology and is often a monetary barrier to adoption of new production systems. Additionally, the use of two simultaneous systems in parallel can provide twice the production capacity with higher turnover rates, minimizing production costs and maximizing output.

The ability to save production time by increasing production turnover and limiting the biological production steps to a single space is highly desirable as both these parameters contribute to lowering the total cost of production (i.e. improved space-time yield). Additionally, vitamin C manufacturers would be able to modify their current production systems without major capital investment, which is a major factor for the adoption of new production pipelines, as the fermenters are already in place. The extra second fermenter system could be simply modified so that two production-scale fermentations could be done simultaneously, further improving yield. Other published technologies attempt to create a one-pot production system by combining both bacteria into a single fermentation vessel. However, this is difficult to control as one bacterium will outcompete the other over time. Consequently, the system needs more frequent, complicated, and expensive monitoring and will have to be reset more frequently, adding cost. The system proposed here avoids these pitfalls since it combines the desired properties of both systems into a single organism.

In providing a solution to a long felt need in the industry, the present inventor has now developed a single bacterial strain able to produce 2-keto-gulonic acid from D-sorbitol, which requires a series of enzymatic conversions not previously provided by any one single microbe. As such, the invention provides in a first aspect a one-pot method for synthesizing L-ascorbic acid (vitamin C). Such a method includes steps of: (a) oxidation of D-sorbitol to L-sorbose by a polyol dehydrogenase; (b) oxidation of the L-sorbose by a sorbose dehydrogenase and sorbosone dehydrogenase to 2-keto-gulonic acid; (c) conversion of the 2-keto-gulonic acid to L-ascorbic acid (vitamin C); and (d) isolation of said L-ascorbic acid (vitamin C) synthesized in the method; wherein: the polyol dehydrogenase, sorbose dehydrogenase, and sorbosone dehydrogenase are expressed by a single, engineered bacterial strain, the bacterial strain solely-capable of the steps (a)-(b) whereby D-sorbitol is oxidized to 2-keto-gulonic acid; and the steps (a)-(b) are carried out in a single fermentation vessel.

In preferred embodiments, the polyol dehydrogenase is polyol dehydrogenase (SldBA) of Gluconobacter oxydans, and the preferred sorbose dehydrogenase and sorbosone dehydrogenase are sorbose dehydrogenase (KVU _2142) and sorbosone dehydrogenase (KVU_0095) of Ketogulonicigenium vulgare.

Engineered bacterial strains according to the invention are preferably an engineered Ketogulonicigenium sp. strain, more preferably a Ketogulonicigenium vulgare strain. It is envisioned that the invention encompasses the use of alternative bacterial species of Ketogulonicigenium including, but not limited to, K. robustum or specific strains including, but not limited to, Ketogulonicigenium sp. DY1, V.6, 291-19, or DYW5.

In certain embodiments, the engineered bacterial strain is a Ketogulonicigenium vulgare strain naturally expressing the sorbose dehydrogenase and sorbosone dehydrogenase, preferably sorbose dehydrogenase (KVU_2142) and sorbosone dehydrogenase (KVU_0095), and engineered to express the polyol dehydrogenase, a heterologous polyol dehydrogenase, preferably a polyol dehydrogenase (SldBA) of Gluconobacter oxydans.

The conversion step (c) is preferably carried out in the same fermentation vessel as steps (a)-(b).

In another aspect, the invention encompasses an engineered Ketogulonicigenium vulgare strain, comprising a heterologous polyol dehydrogenase capable of oxidizing D-sorbitol to L-sorbose.

The engineered Ketogulonicigenium vulgare strain preferably includes the heterologous polyol dehydrogenase (SldBA) of Gluconobacter oxydans, and, more preferably, further expresses a native sorbose dehydrogenase (KVU _2142) and native sorbosone dehydrogenase (KVU _0095), said native dehydrogenases collectively capable of converting L-sorbose to 2-ketogulonic acid. It is further contemplated that the invention is applicable to the use of enzymes having analogous catalytic activities in Ketogulonicigenium sp. including, but not limited to, the sorbose/sorbosone dehydrogenases KVU_0245, KVU_2159, KVU_1366, and KVU_0203, or the sorbosone dehydrogenase KVU_0115.

In yet another aspect, the invention provides a heterologous expression system for oxidation of D-sorbitol to 2-keto-gulonic acid in a one-pot synthesis. Such a system includes a bacterial strain engineered to express a polyol dehydrogenase, a sorbose dehydrogenase, and a sorbosone dehydrogenase, wherein the bacterial strain is solely-capable of oxidizing D-sorbitol to 2-keto-gulonic acid; and a fermentation vessel containing the engineered bacterial strain and operating under fermentation conditions facilitating oxidation of D-sorbitol to 2-keto-gulonic acid by the bacterial strain.

The bacterial strain is preferably an engineered Ketogulonicigenium vulgare strain, wherein, in certain embodiments, the engineered Ketogulonicigenium vulgare strain naturally expresses the sorbose dehydrogenase and sorbosone dehydrogenase and is engineered to express the polyol dehydrogenase, said polyol dehydrogenase being a heterologous polyol dehydrogenase. The heterologous polyol dehydrogenase is preferably from Gluconobacter oxydans.

The exemplary expression system described below utilizes a broad-host-range vector pBBRlp452. This vector is based on the pBBR1MCS vector (Kovach et al. 1994. Biotechniques. 16(5):800-2. PMID: 8068328). The example uses a broad-host-range plasmid since, unlike for E. coli, there is a lack of described plasmids for expression in Ketogulonicigenium, other than pBBRlMCS-2 derivatives. It is envisioned that broad-host-range plasmids capable of being maintained in the Proteobacteria phylum could find use in the present invention. For example, the PromA plasmids, which are well-known broad-host-range plasmids, could be utilized in an alternative system according to the invention. Other useful plasmids include, but are not limited to, pWKS1, RSF1010, R1162, R300B, pCU1, RA3, RK2, RP4, RP1, R6, or pB10.

Expression systems according to the invention may utilize native or non-native promoters functional in Ketogulonicigenium. For constitutive expression in Ketogulonicigenium, the E. coli tufB promoter and the K. robustum orf-01408 (the same as KVU_1695 - perfect homolog) and orf_02221 (the same as KVU_2604 - perfect homolog) promoters have been used in the exemplary embodiment described below. However, any number of E. coli promoters such as the tufB promoter and native Ketogulonicigenium promoters could be utilized in the present invention with only routine experimentation to optimize promoter strength and yield. Inducible promotors could also be utilized in the invention, including those induced by addition of anhydrotetracycline, IPTG, or arabinose, as well as temperature shift induction (e.g. dsrA promoter, derivatives of the pL/Pr promoters, or cspA promoter).

The invention will be more fully understood upon consideration of the following non-limiting Examples.

### III. Examples

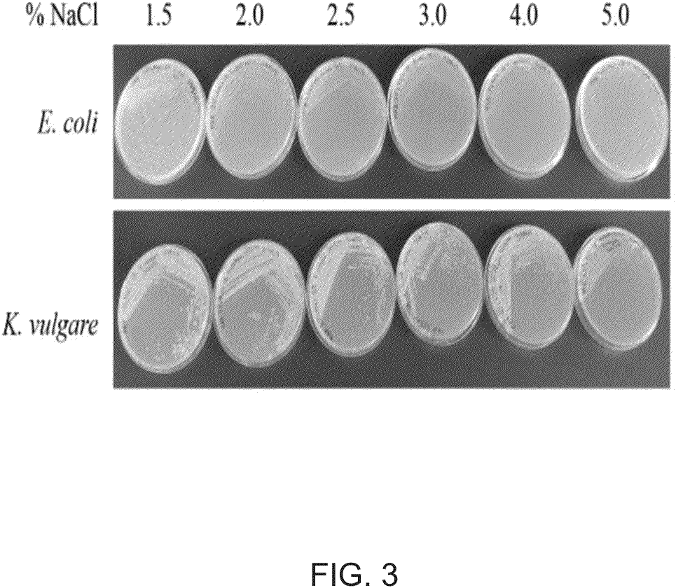

Example 1. Construction of a K. vulgare strain capable of converting D-sorbitol to 2-keto-gulonic acid. An exemplary bacterial strain according to the invention has been prepared and evaluated by the inventor. To construct a strain of K. vulgare able to perform a one-pot synthesis of vitamin C, the sldBA gene from G. oxydans along with the 5′-UTR containing the native promoter was amplified by PCR using a Phusion DNA polymerase. The amplicon was blunt-end cloned into the SspI restriction sites of pBBRlp452 to create pSldBA and was sequenced confirmed. The plasmid was transformed to E. coli S17-1 by electroporation. An overnight culture of K. vulgare and E. coli S17-1 containing pSldBA was mixed 1:1 and the cell pellet washed three times with lysogeny broth (LB). The pellet was resuspended in 100 µl LB and plated as a single spot on LB agar and grown overnight at 37° C. The resulting growth was streaked for isolation on LB agar containing either 4% or 5% NaCl and 50 µg/ml kanamycin to select for K. vulgare containing the plasmid pSldBA (see FIG. 3). Positive transformants were confirmed by colony PCR using primers specific for the sldBA gene. K.vulgare containing pSldBA was further verified by growth on D-sorbitol. Small scale shake flasks were used to determine the production yield of vitamin C. K. vulgare containing pSldBA was grown on D-sorbitol and vitamin C was quantified by iodometric titration after chemical conversion of 2-keto-gulonic acid to ascorbic acid. Briefly, 1-10 ml culture samples were mixed with 3-30 ml 8 M HCl and heated for 30 min at 90° C., after which 4 ml of 5 M NaOH was added to stop the reaction and titrated. Alternatively, vitamin C can be quantified by HPLC after conversion of 2-keto-gulonic acid to vitamin C.

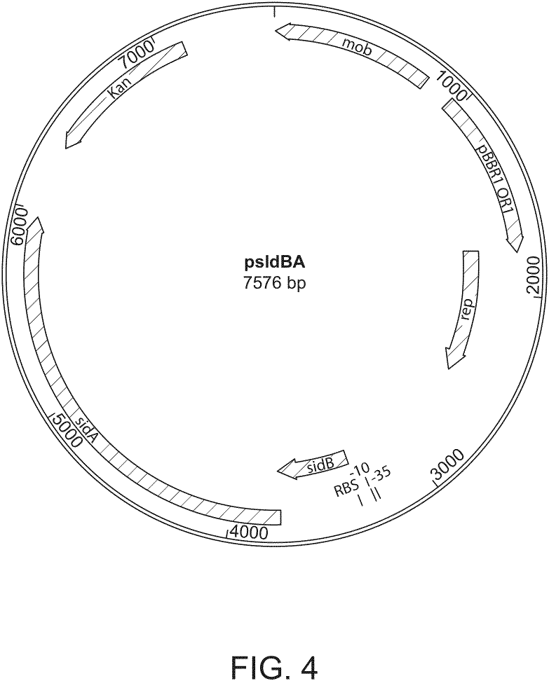

Example 2. Transformation of Ketogulonicigenium vulgare and K. robustum with pSldBA that expresses the sldBA (sorbitol/polyol dehydrogenase) operon from Gluconobacter oxydans. Ketogulonicigenium vulgare and K. robustum were transformed with the broad-host-range plasmid pSldBA that expresses the sldBA operon from Gluconobacter oxydans (FIG. 4). Ketogulonicigenium containing pSldBA was grown on medium containing 20 g/L of either sorbitol or sorbose. Batch cultures were grown for 48h at 28-30° C. and shaking at 250 rpm. The production of 2-keto-L-gulonic acid was assessed by a modified method of Sonoyama et. al. 1981. Briefly, 2-keto-L-gulonic acid was converted to L-ascorbic acid by adding 8 M HCl to in a 1:3 (v/v) ratio with the fermentation broth. This mixture was heated for 30 min at 90° C. with occasional mixing, cooled to room temperature, and used for iodometric titration of vitamin C. When grown on sorbitol yields ranged from 8-12 g/L (40-60% conversion) in 48 h in small scale 100-250 ml cultures. Similar yields were obtained when the production intermediate sorbose was used as the carbon source rather than sorbitol. It is important to note that sorbose is the intermediate of vitamin C production that is produced from the Gluconobacter sorbitol/polyol dehydrogenase.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. Other embodiments and uses of the invention will be apparent to those skilled in the art from consideration from the specification and practice of the invention disclosed herein. It is understood that the invention is not confined to the specific reagents, formulations, reaction conditions, etc., herein illustrated and described, but embraces such modified forms thereof as come within the scope of the following claims.

1. A one-pot method for synthesizing L-ascorbic acid (vitamin C), comprising:
(a) oxidation of D-sorbitol to L-sorbose by a polyol dehydrogenase;
(b) oxidation of the L-sorbose by a sorbose dehydrogenase and sorbosone dehydrogenase to 2-keto-gulonic acid;
(c) conversion of the 2-keto-gulonic acid to L-ascorbic acid (vitamin C); and
(d) isolation of said L-ascorbic acid (vitamin C) synthesized in the method;
wherein: the polyol dehydrogenase, sorbose dehydrogenase, and sorbosone dehydrogenase are expressed by a single, engineered bacterial strain, the bacterial strain solely-capable of the steps (a)-(b) whereby D-sorbitol is oxidized to 2-keto-gulonic acid; and the steps (a)-(b) are carried out in a single fermentation vessel.

2. The method according to claim 1, wherein the polyol dehydrogenase is polyol dehydrogenase (SldBA) of Gluconobacter oxydans.

3. The method according to claim 1, wherein the sorbose dehydrogenase and sorbosone dehydrogenase are sorbose dehydrogenase (KVU_2142) and sorbosone dehydrogenase (KVU 0095) of Ketogulonicigenium vulgare.

4. The method according to claim 1, wherein the polyol dehydrogenase is polyol dehydrogenase (SldBA) of Gluconobacter oxydans, and the sorbose dehydrogenase and sorbosone dehydrogenase are sorbose dehydrogenase (KVU_2142) and sorbosone dehydrogenase (KVU_0095) of Ketogulonicigenium vulgare.

5. The method according to claim 1, wherein the engineered bacterial strain is an engineered Ketogulonicigenium sp. strain.

6. The method according to claim 1, wherein the engineered bacterial strain is an engineered Ketogulonicigenium vulgare strain.

7. The method according to claim 1, wherein said engineered bacterial strain is a Ketogulonicigenium vulgare strain naturally expressing the sorbose dehydrogenase and sorbosone dehydrogenase and engineered to express the polyol dehydrogenase, wherein said polyol dehydrogenase is a heterologous polyol dehydrogenase.

8. The method according to claim 7, wherein the heterologous polyol dehydrogenase is polyol dehydrogenase (SldBA) of Gluconobacter oxydans.

9. The method according to claim 7, wherein the sorbose dehydrogenase is sorbose dehydrogenase (KVU_2142) and the sorbosone dehydrogenase is sorbosone dehydrogenase (KVU_0095).

10. The method according to claim 1, wherein the conversion step (c) is carried out in the same fermentation vessel as steps (a)-(b).

11. An engineered Ketogulonicigenium vulgare strain, comprising a heterologous polyol dehydrogenase capable of oxidizing D-sorbitol to L-sorbose.

12. The engineered Ketogulonicigenium vulgare strain according to claim 11, wherein the heterologous polyol dehydrogenase is polyol dehydrogenase (SldBA) of Gluconobacter oxydans.

13. The engineered Ketogulonicigenium vulgare strain of claim 11, wherein said strain expresses a native sorbose dehydrogenase (KVU_2142) and native sorbosone dehydrogenase (KVU_0095), said native dehydrogenases collectively capable of converting L-sorbose to 2-ketogulonic acid.

14. The engineered Ketogulonicigenium vulgare strain according to claim 11 wherein the engineered Ketogulonicigenium vulgare strain is used for a one-pot synthesis of L-ascorbic acid (vitamin C).

15. (canceled)

16. (canceled)

17. A heterologous expression system for oxidation of D-sorbitol to 2-keto-gulonic acid in a one-pot synthesis, comprising a bacterial strain engineered to express a polyol dehydrogenase, a sorbose dehydrogenase, and a sorbosone dehydrogenase, wherein the bacterial strain is solely-capable of oxidizing D-sorbitol to 2-keto-gulonic acid; and a fermentation vessel containing the engineered bacterial strain and operating under fermentation conditions facilitating oxidation of D-sorbitol to 2-keto-gulonic acid by the bacterial strain.

18. The heterologous expression system according to claim 17, wherein the bacterial strain is an engineered Ketogulonicigenium sp. strain.

19. The heterologous expression system according to claim 18, wherein the engineered Ketogulonicigenium sp. strain naturally expresses the sorbose dehydrogenase and sorbosone dehydrogenase and is engineered to express the polyol dehydrogenase, said polyol dehydrogenase being a heterologous polyol dehydrogenase.

20. The heterologous expression system according to claim 19, wherein the heterologous polyol dehydrogenase is from Gluconobacter oxydans.

21. The heterologous expression system according to claim 18, wherein the Ketogulonicigenium sp. is a Ketogulonicigenium vulgare strain.
